SEQUENCE LISTING (3 sequences)

SEQ ID NO: 1 - protein, 803 aa, Clostridium Difficile
  Protein is designated Cwp84
MRKYKSKKLSKLLALLTVCFLIVSTIPVSAENHKTLDGVETAEYSESYLQYLEDVKNGDTAKYNGVIPFPHEMEGTTLRNKGRSSLPSAYKSSVAYNPMDLGLTTPAKNQGSLNTCWSFSGMSTLEAYLKLKGYGTYDLSEEHLRWWATGGKYGWNLDDMSGSSNVTAIGYLTAWAGPKLEKDIPYNLKSEAQGATKPSNMDTAPTQFNVTDVVRLNKDKETVKNAIMQYGSVTSGYAHYSTYFNKDETAYNCTNKRAPLNHAVAIVGWDDNYSKDNFASDVKPESNGAWLVKSSWGEFNSMKGFFWISYEDKTLLTDTDNYAMKSVSKPDSDKKMYQLEYAGLSKIMSNKVTAANVFDFSRDSEKLDSVMFETDSVGAKYEVYYAPVVNGVPQNNSMTKLASGTVSYSGYINVPTNSYSLPKGKGAIVVVIDNTANPNREKSTLAYETNIDAYYLYEAKANLGESYILQNNKFEDINTYSEFSPCNFVIKAITKTSSGQATSGESLTGADRYETAVKVSQKGWTSSQNAVLVNGDAIVDALTATPFTAAIDSPILLTGKDNLDSKTKAELQRLGTKKVYLIGGENSLSKNVQTQLSNMGISVERISGSDRYKTSISLAQKLNSIKSVSQVAVANGVNGLADAISVGAAAADNNMPIILTNEKSELQGADEFLNSSKITKSYIIGGTATLSSNLESKLSNPTRLAGSNRNETNAKIIDKFYPSSDLKYAFVVKDGSKSQGDLIDGLAVGALGAKTDSPVVLVGNKLDESQKNVLKSKKIETPIRVGGNGNESAFNELNTLLGK

SEQ ID NO: 2 - protein, 290 aa, Clostridium difficile
  Protein is designated FliC
MRVNTNVSALVANNQMGRNVNAQSKSMEKLSSGVRIKRAADDAAGLAISEKMRAQIKGLDQAGRNVQDGISVVQTAEGALEETGNILQRMRTLSLQSSNETNTAEERQKVADELLQLKDEVERISSSIEFNGKKLLDGSSTEIRLQVGANFGTNVAGTTNNNNEIKVALVNTSSIMSKAGITSSTIASLNVDGASGTDAAKQMVSSLDMALKELNTSRAKLGAQQNRLESTQNNLNNTIENVTAAESRIRDTDVASEMVNLSKMNILVQASQSMLAQANQQPQGVLQLLG

SEQ ID NO: 3 - protein, 506 aa, Clostridium difficile
  Protein is designated FliD
MSSISPVRVTGLSGNFDMEGIIEASMIRDKEKVNKAKQDQQIVKWKQEIYRDIIKESKNLYDKYLNGDSPNSITNKKAYSATRITSSDESIIVAKGSAGAEKINYQFAVSQMAEPAKVTIKLNSSDPIVQQFPPNASGASSLNIGGVNIPISEQDTTSTIVSKINSLCADNDIRASYSEMTGELIISRKQTGSSSDIDLKVIGNDSLAGQIASDNGITFTTDASGTKSAVVYGKNLEADVTDDQGRVTHISKEQNSFKIDNIDYNVNSKGSAKLVSVTDTEEATKNMKAFVDDYNALMDKVYGLVTTKKSKDYPPLTDEQKDDMTTEEIEKWEKKAKEGILRNDDELRAFVEDIQSMFFGDADTIIALRKLGISEHENYNKKGQISFNADTFSKALIDDSDKVYKALAGYSSNYDDKGMFEKLKKIVFEYSGSSASKLTKKAGMENSSSASQNVYSKQIAEQERNISRLVEKMNDKEKRLYAKYSALESLLNKYSSQMNYFSQAQG

(19) United States
(12) Patent Application Publication
(10) Pub. No.: US 2011/0020356 A1
(43) Pub. Date: Jan. 27, 2011

(54) Therapeutic clostridium difficile antibody compositions

(76) Inventors: Lin Fang, Winnipeg (CA); Ronald R. Marquardt, Winnipeg (CA); R. Terence Sellen, Oak Bank (CA)

Correspondence Address: Robert W.B. Bailey, Box 2, Station L., Winnipeg, MB, R3H 0Z4, CA

(21) Appl. No.: 12/923,394

(22) Filed: Sep. 20, 2010

Related U.S. Application Data
(63) Continuation-in-part of application No. 12/000,020, filed on Dec. 7, 2007.

(51) Int. Cl. A61K 39/395; C07K 16/40; C07K 16/12; A61P 31/04

(52) U.S. Cl. 424/139.1; 530/387.9

(57) ABSTRACT

The C. difficile proteins Cwp84, FliC and FliD, known to have conserved peptide sequences, were separately injected into female chickens, and the antibody rich egg yolks harvested. The antibody compositions were then titered by ELISA and western immunoblotting. Anti-FliD IgY, anti-Cwp84 IgY, and an equititer cocktail of the three IgY antibodies was administered to hamsters infected with a C. difficile strain and found effective as potential treatments.

The sequence listing in this application is identical to that filed in Ser. No. 12/000,020, 7 Dec. 2007, as (Copy 1 Replacement and Copy 2 Replacement) on floppy disks, each containing the file named “sequence listing.txt” which is 13,757 bytes (measured in MS-DOS Windows 95 and 98) created on Apr. 23, 2008, together with a paper copy of the sequence listing. Applicant according requests that the sequence listing of Ser. No. 12/000,020, which is compliant with regulation, be considered as if filed with this application, and that the sequence listing be herein incorporated by reference.

### FIELD OF INVENTION

This invention is directed to antibody compositions against bacteria, specifically including Clostridium difficile. C. difficile is a well known primary infective agent causing iatrogenic diarrhea in hospitals, and is thought widespread in livestock in animal husbandry. C. difficile is increasingly resistant to antibiotics. These antibodies are generated by surface proteins (antigens) of C. difficile. The proteins (antigens) are purified before they are used to generate the antibodies. Although the precise nature of antibodies is probably unimportant, whether monoclonal, or polyclonal, IgG or IgY, the antibody compositions of the invention are anti-Cpw84 IgY, anti-FliC IgY, anti-FliD IgY, from chickens, which applicants previously tested in vitro.

### BACKGROUND

C. difficile has a number of characteristic proteins, the so-called surface proteins, one of which is SlpA, often referred to as P36 and P47, which are produced by a single gene as a single protein and then split, another is FliC (the main flagellum component), another is FliD (the flagellum tip or cap protein), another is Cwp84 (a cell wall protein, thought a cysteine protease and perhaps essential to the bacterial metabolism), Cwp66 (another cell wall protein), and Fbp68 (an associated cell wall protein). These surface proteins are distinct from toxins A and B, which are also characteristic proteins generated by C. difficile, but are not integral to its structure.

For an antibody composition to be effective against a bacteria, it must interact with a protein, usually an outermembrane protein or a toxin, specific to that bacteria. Although this begs the question since bacteria are to some extent defined by their specific proteins, themselves expressed by bacterial DNA sequences, the composition, containing one or more antibodies must interact with a particular protein of the organism. Although proteins have a huge number of sequential aminoacid possibilities, as indicated below, most proteins having a particular function in a particular bacteria will have one or more common sequences of aminoacids. These sequences are known as conserved sequences, and should be present in every protein of this type in the species of bacteria. When a protein is used as an antigen to generate antibodies it is not necessary to use the entire protein; it is possible to use a fragment of the protein capable of generating an immune response, the fragment must form a conserved sequence of the sequence to be effective.

### PRIOR ART

As noted above C. difficile has six surface proteins, which were considered. US Patent Application Publication 2003/0054009, to Windle et al., 20 Mar. 2003, teaches two SlpA sequences (No. 1 and 2), one for each SlpA protein, suitable to generate vaccines. Further taught are six sequences (No. 3, 4, 5, 6, 7 and 8) for the entire SlpA before cleavage for six strains of C. difficile. It was intended either to introduce the antigen (protein) itself to directly generate antibodies in the cell, or to introduce DNA which then generates the antigen, which indirectly generates antibodies in the cell. Windle did not inject either DNA or proteins as a vaccine into human patients, to generate antibodies. What Windle did was to test antigen peptide SEQ ID No. 1, and SEQ ID No. 2 against individuals who had recovered from C. difficile infections, and showed that these induced a strong immunogenic response, demonstrating that antibodies to these sequences were present. This was carried out by introducing peptide sequences into sera from patients (page 3, paragraph [0068], page 4 paragraph [0090], page 5 paragraph [0097]) showing that antibodies to these antigens were present. Consequently Windle concluded that these sequences were potential vaccines, however this was not tested.

More recently Pechine et al. tested 17 isolates from infected human patients, the proteins tested were FliC, FliD, Cwp66, both C and N terminal domains, and Cwp84, N terminal. Details of the generation of protein sequences, but not the actual sequences are given. FliC and FliD were detected (by antibody reaction) in 15 of 17 isolates, N-terminal Cwp66 was detected in all isolates, C-terminal Cwp66 was detected in 12 of 17 isolates, Cwp84 was detected in all isolates. That is the antigens (proteins) were detected in the isolates by antibodies. It was confirmed that there is relatively little genetic variability in FliC and FliD, Cwp66 showed considerable genetic variation, especially at the C-terminal, while Cwp84 appeared uniform. Pechine et al. state that SlpA proteins are useful for phenotyping C. difficile, that is distinguishing genetic strains, thus not for use as antigens. The cwp66 gene (as opposed to Cwp66 protein) both 5′ and 3′ parts show high variability, while the 3′ part is known to be highly variable also, and thus the Cwp66 protein is suitable material for genetic analysis, and not for use as an antigen.

Pechine et al., conclude that Cwp84, FliC, and FliD proteins, or aminoacid (peptide) sequences thereof may be suitable antigens against C. difficile, which can be incorporated into a vaccine. No vaccine was generated. No vaccine was tested, so the effectiveness of vaccines containing Cwp84, FliC, and FliD proteins as antigens was unknown. Certainly Pechine et al., did not use the suggested vaccines to generate antibodies in animals as a treatment against C. difficile infection. There is no clinical proof of any possible effectiveness.

More generally the proteins FliC, FliD, Fbp68, SlpA, Cwp66, and Cwp84 are believed to be implicated in C. difficile's colonization strategy, in the human colon. This belief is based on the fact the convalescent sera of human CDI (C. Difficile Infection), that is sera from human patients who survived the infection, have antibodies to these proteins. C. difficile infects by adhering to mucosal cells in the human colon (gut). The role of C. difficile's toxins A and B and its surface proteins FliC, FliD, Fbp68, SlpA, Cwp66, and Cwp84 in this process, is uncertain.

It is noteworthy that subjects asymptomatically colonized by C. difficile, show decreased risk of later CDI. This was tested by pre-colonizing clindamycin treated hamsters with non-toxigenic strains of C. difficile, which were protected when tested by a wild known toxigenic strain of C. difficile.

Previously colonization factors specific egg yolk IgY antibodies (including FliC, FliD, Fbp68, SlpA, Cwp66, and Cwp84) had not been thought effective against CDI or recurring or chronic CDI, either alone or with toxin-specific IgY (including toxins A and B. IgY refers to immunoglobulin (antibodies) from avian species, typically chickens, IgG refers to immunoglobulin (natibodies) from mammals, typically rabbits.

Both SlpA and Cwp66, were considered too variable to generate effective antibodies against all strains of C. difficile, while there was no indication that Fbp68 would be effective. Applicants therefore decided to generate antibodies in avian egg yolk, by injecting the proteins Cwp84, FliC and FliD into hens. The egg yolks were processed to provide separate antibody compositions which were then tested in vitro against C. difficile cultures, and were found effective. Details are given in U.S. patent application Ser. No. 12/000,020, filed 7 Dec. 2007, published as US Published Patent Application 2008/0222739, 11 Sep. 2008, hereby incorporated by reference. In this publication, anti-FliD IgY was significant at the 1% level, anti-Cwp84 IgY at the 5% level, anti-FliC IgY at between 5% and 10% level, all by Student's t test, which indicated that these might generate effective antibody compositions for in vivo testing. Later experiment demonstrated that anti-FliC IgY, anti-FliD IgY, anti-Cwp84 IgY, and pooled (mixed anti-FliC IgY, anti-FliD IgY and anti-Cwp84 IgY) had a significant difference from IgY from unimmunized hens. In this experiment the effectiveness was FliC>Cwp84>Pooled>FliD.

Advantages of using egg yolk IgY are that the antibodies can be manufactured on an industrial scale. They are easily purified in large quantity from the egg yolks; less antigen (protein) is required to produce efficient, sustained, immune response in the chickens; IgY production is less invasive and less stressful on the birds; the production costs are low; and the product is safe for human ingestion. Mammalian antibodies are much harder to produce in quantity and may contain heat insensitive infective agents, such as bovine spongiform encephalopy. Oral application/ingestion of egg yolk IgY is easy and presents no difficulties. Further prolonged use of the antibodies in hospital treatment of C. difficile will not generate resistance to the antibodies, unlike the prolonged use of antibiotics.

In practice C. difficile is difficult to vaccinate against since it operates against (or in) the gut lining of mammals and humans, and this zone is remote from the bloodstream of the animal. That is antibodies in the animal's bloodstream do not interact with C. difficile directly and thus do not easily affect the infection, which continues unabated. Vaccination is not a practical solution. Mucosal immunity is needed as C. difficile colonizes and destroys mucosal cells. Even if vaccination was effective it is too slow, taking two or three weeks to generate sufficient antibodies. The patient will die or recover in that time. The oral antibody composition can be ingested immediately by the patient, whether human or livestock, with immediate rapid response.

As the above three proteins, Cwp84, FliC, and FliD are surface proteins, and homogenous, they are exposed wherever C. difficile operates. Thus antibodies thereto can be expected to interact with it on contact. As this is acknowledged to be easier in the intestine, use of these antibodies in therapeutic compositions, orally dispensed in food or drink ingested by the animal or human, seemed feasible. Antibodies for internal proteins are less likely to be effective.

It was therefore decided to generate antibodies in avian egg yolk, by injecting the proteins Cwp84, FliC and FliD into bird species, typically hens. As will be understood by those skilled in the art, selected sequences from these proteins could possibly also be used as antigens to generate effective antibodies. The antibody compositions were tested on experimental animals, for effectiveness, in vivo. It is therefore a principal object of the invention to provide antibody compositions comprising antibodies selected from the group consisting of anti-FliC Igy, anti-Flid IgY, and anti-Cwp84 IgY, and mixtures thereof. It is another principal object of the invention to treat mammals infected by Clostridium difficile by compositions comprising antibodies selected from the group consisting of anti-FliC Igy, anti-Flid IgY, and anti-Cwp84 IgY, and mixtures thereof. Other objects of the invention will be apparent to those skilled in the art from the following specification and appended claims.

### DESCRIPTION OF THE INVENTION

Full protein sequences are given in the appendices, for Cwp84 (Sequence No. 1) FliC (Sequence No. 2) and FliD (Sequence No. 3). As will be understood by those skilled in the art, these sequences are the entire aminoacid sequence for samples of these C. difficile proteins, that is other similar but not identical sequences may also be defined as Cwp84, FliC, FliD proteins, however the differences are generally thought small, insignificant and negligible in their effects.

These three proteins are generated by known developed scientific techniques until a sufficient amount of each protein is available, which is then injected singly by known techniques into female chickens (hens). The hens develop specific antibodies to the proteins, which inter alia are found in the yolks of eggs laid by the hens. The eggs and their yolks are then harvested. The yolks are then treated to provide an antibody rich composition, suitable for use in the passive control of C. difficile. These compositions were then tested against C. difficile as indicated in the experimental data and found to have statistically significant effects. The next stage would be to add these compositions in suitable batches to food, liquid or solid, which then is then orally ingested by a patient, human or animal (usually, but not exclusively, in the sense of terrestrial mammal), and assess the clinical results, to determine the in vivo effects. One or more antibody compositions may be combined under these circumstances as an effective treatment.

As known to those skilled in the art, the polypeptide preferably consists of the entire sequence of Cwp84, FliC or FliD, or at least comprises the entire sequence of Cwp84, FliC, or FliD, thus guaranteeing the presence of every aminoacid in the sequence. Often satisfactory antibodies can be generated using a protein having an aminoacid sequence substantially identical to that of a particular protein. Sometimes effective antibodies can be generated by proteins having at least about 60% or 70% homology with a particular protein, that is at least 60 or 70% of the aminoacids are identical and in the same relative positions or order. Further sometimes a homologue, analogue or derivative of these proteins will generate effective vaccines. It must be understood that effective proteins must include conserved sequences which themselves are immunogenic fragments, and it may be difficult to identify such effective fragments without experiment.

C. difficile's general method of infection is by oral ingestion and attacking through and infesting the gut wall. Injection of a vaccine, into vein or muscle, will not usually cause antibodies generated by the vaccine to reach the site of infection which is the inflamed, damaged or destroyed gut wall remote from blood vessels in time or quantity to halt the infection. Feeding patients the antibodies in food will both effectively prevent C. difficile from colonizing the gut wall, and neutralize C. difficile in the process of colonization, by binding the proteins Cwp84, FliC, FliD and thus rendering them ineffective. It is believed that the six surface proteins noted above, also referred to as adhesion factors, or colonization factors, function to attach to the gut wall, and that C. difficile being unable to do so in the presence of antibodies will be unable to infect patients.

It is thought that the above noted proteins Cwp84, FliC, FliD, are present in other Clostridium species, and possibly other bacterial genera. If so the antibodies developed will be of broader effect and significance than C. difficile, alone.

In one broad aspect the invention is directed to an antibody composition comprising at least one IgY egg yolk component, selected from group consisting of anti-Cwp84 IgY, anti-FliC IgY, anti-FliD IgY, each said IgY egg yolk component being produced by injecting chickens separately with a different antigen, said antigens being the proteins, Cwp84, FliC, and FliD of C. difficile. Preferably Cwp84 has the aminoacid sequence of Sequence No. 1, and FliC has the aminoacid sequence of Sequence No. 2, and FliD has the aminoacid sequence of Sequence No. 3. More preferably the IgY component comprises anti-FliD IgY, which may be purified anti-FliD IgY, or may be purified anti-FliD IgY additionally comprising natural egg yolk. The IgY component may instead comprise anti-Cwp84 IgY. The IgY component may be a mixture of anti-Cwp84 IgY, anti-FliC IgY, and anti-FliD IgY, which may have approximately equal antibody titers. Preferably the antibody composition comprises a pharmaceutical carrier.

In a second broad aspect the invention is directed to a method of treatment of lower mammals having a C. difficile infection, comprising administering an antibody composition 20′ comprising at least one IgY egg yolk component, selected from group consisting of anti-Cwp84 IgY, anti-FliC IgY, anti-FliD IgY, each said IgY egg yolk component being produced by injecting chickens separately with a different antigen, said antigens being the proteins, Cwp84, FliC, and FliD of C. difficile. Preferably Cwp84 has the aminoacid sequence of Sequence No. 1, and FliC has the aminoacid sequence of Sequence No. 2, and FliD has the aminoacid sequence of Sequence No. 3. More preferably the IgY component comprises anti-FliD IgY, which may be purified anti-FliD IgY, or may be anti-FliD IgY comprising natural egg yolk. The IgY component may instead comprise anti-Cwp84 IgY. The IgY component may be a mixture of anti-Cwp84 IgY, anti-FliC IgY, and anti-FliD IgY, which may have approximately equal antibody titers. Preferably the antibody composition comprises a pharmaceutical carrier.

### DESCRIPTION OF THE PREFERRED EMBODIMENTS

The proteins Cwp84, FliC, FliD prepared by standard techniques were injected using known techniques into hens (female chickens) to create antibodies of the IgY type. The eggs of the chickens were then harvested. The egg yolks were then separated and dried and tested for the presence of antibodies. Typically the antibodies of the dried egg yolk antibody composition were purified or concentrated by pH precipitation, substantially separating egg yolk and antibodies. The purified antibody composition was then tested as indicated below against C. difficile.

Eggs from immunized Leghorn chickens were used to prepare lyophilized egg yolk preparations containing Cwp84, FliC, and FliD CF (colonization factor)-specific egg yolk antibodies. The presence and titers of the C. difficile CF specific IgY antibodies was determined by the ELISA and Western immunoblotting techniques. The efficacy of these antibodies in preventing C. difficile mediated morbidity and mortality was subsequently investigated in Syrian hamsters, which are the internationally accepted standard. If successful the trials would indicate that further testing for livestock and humans was warranted.

In experiment 1, male Syrian hamsters, 80 to 100 g, were were pre-treated by gavage with clindamycin phosphate, 30 mg/kg, 5 days prior to adminstering (by gavage) 1×10³ C. difficile strain 630 spores admixed with a 1:1:1 mixture of the CF-specific egg yolk preparations, or purified IgY antibodies prepared from these egg yolks. The mixtures were essentially equi-titers of each antibody. The animals then received the mixture of CF-specific egg yolk preparations or purified IgY antibodies every 24 hours thereafter. Control groups received either 0.1M carbonate buffer (pH 9.5) (used to rehydrate the lyophilized egg yolk preparations) or an egg yolk preparation produced from eggs laid by unimmunized chickens. Beginning 48 hours post-infection and for 11 days thereafter, the animals were closely monitored every 4 hours for signs of C. difficile infection, including the appearance of peri-anal fecal staining and/or lethargy. Animals exhibiting these signs were immediately euthanized by CO₂ asphyxia. The results showed that hamsters treated with the 1:1:1 cocktail C. difficile CF-specific egg yolk preparations or a purified CF-specific IgY preparation were significantly protected from C. difficile strain 630 infection relative to control animals treated with carbonate buffer or an egg yolk preparation produced from unimmunized chicken eggs. The result suggests that egg yolk preparations obtained from chickens immunized with recombinant C, difficile CFs may represent an effective, safe and cost-effective treatment option in humans suffering from CDI (C. difficile infection).

Survival Table I for Hamsters

| | Carbonate | Control | Toxin A/B | C/D/W | Clinda- |
|---|---|---|---|---|---|
| | Buffer | IgY | IgY | IgY | mycin Only |
| Hours | N = 4 | N = 8 | N = 8 | N = 8 | N = 2 |
| 0 | 100% | 100% | 100% | 100% | 100% |
| 41 | 100% | 1005 | 100% | 100% | 100% |
| 45 | 75% | 62.5% | 87.5% | 100% | 100% |
| 49 | 75% | 62.5% | 87.5% | 100% | 100% |
| 53 | 75% | 37.5% | 87.5% | 87.5% | 100% |
| 57 | 50% | 37.5% | 62.5% | 87.5% | 100% |
| 61 | 25% | 25% | 37.5% | 75% | 100% |
| 65 | 0% | 12.5% | 37.5% | 62.5% | 100% |
| 69 | 0% | 12.5% | 37.5% | 37.5% | 100% |
| 73 | 0% | 0% | 25% | 37.5% | 100% |
| 77 | 0% | 0% | 12.5% | 37.5% | 100% |
| 81 | 0% | 0% | 12.5% | 25% | 100% |
| 85 | 0% | 0% | 12.5% | 25% | 100% |
| 89 | 0% | 0% | 0% | 12.5% | 100% |
| 93 | 0% | 0% | 0% | 12.5% | 100% |

The anti-bodies are control IgY that is natural egg yolk without C. difficile antibodies; Toxin A/B IgY, which is an equititer of anti-Toxin A IgY and anti-Toxin B IgY (these antibodies to the C. difficile Toxins A and B are known); C/D/W IgY is the 1:1:1 equititer mixture of C. difficile anti-FliC IgY, anti-FliD IgY, and anti-Cwp84 IgY. As can be seen the triple coktail of FliC-FliD-Cwp84 IgY was effective in treating C. difficile in hamsters.

In experiment 2, a similar approach was followed, male Syrian hamsters, 80 to 100 g, were were pre-treated by gavage with clindamycin phosphate, 30 mg/kg, 5 days prior to adminstering (by gavage) 1×10³ C. difficile strain 630 spores. Treatments were then givne by oral gavage in 0.1M carbonate buffer (pH 9.0) once daily for 10 days commencing on day 5 (the same day spores given). The hamsters were closely monitored every 4 hours for signs of C. difficile infection, including the appearance of peri-anal fecal staining and/or lethargy. Animals exhibiting these signs were immediately euthanized by CO₂ asphyxia.

Survival Table II for Hamsters

| | FliD IgY | FliD | C/D/W | Cwp84 | Control | Clinda- |
|---|---|---|---|---|---|---|
| | Egg yolk | IgY | IgY | IgY | Egg Yolk | mycin Only |
| Days | N = 8 | N = 8 | N = 8 | N = 8 | N = 8 | N = 2 |
| 0 | 100% | 100% | 100% | 100% | 100% | 100% |
| 1 | 100% | 100% | 100% | 100% | 100% | 100% |
| 2 | 100% | 100% | 100% | 100% | 100% | 100% |
| 3 | 87.5% | 75% | 87.5% | 87.5% | 87.5% | 100% |
| 4 | 87.5% | 50% | 50% | 25% | 62.5% | 100% |
| 5 | 75% | 37.5% | 25% | 25% | 37.5% | 100% |
| 6 | 75% | 37.5% | 25% | 25% | 12.5% | 100% |
| 7 | 75% | 37.5% | 25% | 25% | 12.5% | 100% |
| 8 | 75% | 37.5% | 25% | 25% | 12.5% | 100% |
| 9 | 75% | 37.5% | 25% | 25% | 12.5% | 100% |

The anti-bodies are anti-FliD IgY in natural egg yolk; anti-FliD IgY alone; C/D/W IgY is the 1:1:1 mixture of anti-FliC IgY, anti-FliD IgY, and anti-Cwp84 IgY; anti-Cwp84 IgY; control egg yolk. As can be seen anti-FliD IgY in egg yolk was superior to anti-FliD IgY alone, itself better than the triple cocktail of anti-FliC IgY, anti-FliD IgY, and anti-Cwp84 IgY, or anti-Cwp84 IgY alone, but all were effective in treating C. difficile in hamsters.

Further tests using the same protocol were performed with the total survival rates as shown:

| | |
|---|---|
| Clindamycin phosphate without C. difficile | 10/10 |
| Clindamycin phosphate C. difficile in carbonate buffer | 2/12 |
| Clindamycin phosphate C. difficile with control egg yolk | 11/48 |
| Clindamycin phosphate C. difficile anti-FliD IgY | 12/16 |
| in control egg yolk | |
| Clindamycin phosphate C. difficile anti-FliD IgY alone | 14/23 |
| in carbonate buffer | |
| Clindamycin phosphate C. difficile C/D/W IgY cocktail | 27/46 |
| in egg yolk | |

The significant differences compared to Clindamycin phosphate plus control egg yolk 11/48 survival, were:

p=0.00052 for clindamycin phosphate C. difficile anti-FliD IgY in control egg yolk;
p=0.0031 for clindamycin phosphate C. difficile anti-FliD IgY in carbonate buffer;
p=0.00068 for clindamycin phosphate C. difficile C/D/W IgY cocktail in egg yolk. As those skilled in the art are aware these results are highly statistically significant, as tested by the two tailed Fisher Exact test, in showing the effectiveness of the antibodies against CDI (C. difficile infection). Anti-FliD IgY will neutralize the pathogenic C. difficile. The effectiveness of the antibodies in hamsters can be taken as indicative of their effectiveness in mammals in general, lower mammals in particular, and possibly humans. Further clinical experiment is indicated.

Natural or control egg yolk, refers to egg yolk from unimmunized hens.

As those skilled in the art would realize these prefer'red described details and materials and components can be subjected to substantial variation, modification, change, alteration, and substitution without affecting or modifying the function of the described embodiments.

Although embodiments of the invention have been described above, it is not limited thereto, and it will be apparent to persons skilled in the art that numerous modifications and variations form part of the present invention insofar as they do not depart from the spirit, nature and scope of the claimed and described invention.

APPENDIX I
Single Letter Amino Acid Code

Sequence No. 1
(Cwp84)
MRKYKSKKLSKLLALLTVCFLIVSTIPVSAENHKTLDGVETAEYSESYLQ
YLEDVKNGDTAKYNGVIPFPHEMEGTTLRNKGRSSLPSAYKSSVAYNPMD
LGLTTPAKNQGSLNTCWSFSGMSTLEAYLKLKGYGTYDLSEEHLRWWATG
GKYGWNLDDMSGSSNVTAIGYLTAWAGPKLEKDIPYNLKSEAQGATKPSN
MDTAPTQFNVTDVVRLNKDKETVKNAIMQYGSVTSGYAHYSTYFNKDETA
YNCTNKRAPLNHAVAIVGWDDNYSKDNFASDVKPESNGAWLVKSSWGEFN
SMKGFFWISYEDKTLLTDTDNYAMKSVSKPDSDKKMYQLEYAGLSKIMSN
KVTAANVFDFSRDSEKLDSVMFETDSVGAKYEVYYAPVVNGVPQNNSMTK
LASGTVSYSGYINVPTNSYSLPKGKGAIVVVIDNTANPNREKSTLAYETN
IDAYYLYEAKANLGESYILQNNKFEDINTYSEFSPCNFVIKAITKTSSGQ
ATSGESLTGADRYETAVKVSQKGWTSSQNAVLVNGDAIVDALTATPFTAA
IDSPILLTGKDNLDSKTKAELQRLGTKKVYLIGGENSLSKNVQTQLSNMG
ISVERISGSDRYKTSISLAQKLNSIKSVSQVAVANGVNGLADAISVGAAA
ADNNMPIILTNEKSELQGADEFLNSSKITKSYIIGGTATLSSNLESKLSN
PTRLAGSNRNETNAKIIDKFYPSSDLKYAFVVKDGSKSQGDLIDGLAVGA
LGAKTDSPVVLVGNKLDESQKNVLKSKKIETPIRVGGNGNESAFNELNTL
LGK
Sequence No. 2
(FliC)
MRVNTNVSALVANNQMGRNVNAQSKSMEKLSSGVRIKRAADDAAGLAISE
KMRAQIKGLDQAGRNVQDGISVVQTAEGALEETGNILQRMRTLSLQSSNE
TNTAEERQKVADELLQLKDEVERISSSIEFNGKKLLDGSSTEIRLQVGAN
FGTNVAGTTNNNNEIKVALVNTSSIMSKAGITSSTIASLNVDGASGTDAA
KQMVSSLDMALKELNTSRAKLGAQQNRLESTQNNLNNTIENVTAAESRIR
DTDVASEMVNLSKMNILVQASQSMLAQANQQPQGVLQLLG
Sequence No. 3
(FliD)
MSSISPVRVTGLSGNFDMEGIIEASMIRDKEKVNKAKQDQQIVKWKQEIY
RDIIKESKNLYDKYLNGDSPNSITNKKAYSATRITSSDESIIVAKGSAGA
EKINYQFAVSQMAEPAKVTIKLNSSDPIVQQFPPNASGASSLNIGGVNIP
ISEQDTTSTIVSKINSLCADNDIRASYSEMTGELIISRKQTGSSSDIDLK
VIGNDSLAGQIASDNGITFTTDASGTKSAVVYGKNLEADVTDDQGRVTHI
SKEQNSFKIDNIDYNVNSKGSAKLVSVTDTEEATKNMKAFVDDYNALMDK
VYGLVTTKKSKDYPPLTDEQKDDMTTEEIEKWEKKAKEGILRNDDELRAF
VEDIQSMFFGDADTIIALRKLGISEHENYNKKGQISFNADTFSKALIDDS
DKVYKALAGYSSNYDDKGMFEKLKKIVFEYSGSSASKLTKKAGMENSSSA
SQNVYSKQIAEQERNISRLVEKMNDKEKRLYAKYSALESLLNKYSSQMNY
FSQAQG

APPENDIX II
Three Letter Amino Acid Code

| | |
|---|---|
| <110> | FANG, Lin |
| | MARQUARDT, Ronald |
| | SELLEN, Robert Terence |
| <120> | Therapeutic Clostridium Difficile Antibody Compositions |
| <130> | 428-2US |
| <140> | N/A |
| <150> | N/A |
| <151> | N/A |
| <160> | 3 |
| <170> | WordPerfect 6.0 converted ASCII |
| <210> | 1 |
| <211> | 813 |
| <212> | PRT |
| <213> | Clostridium Difficile |
| <220> | |
| <223> | Protein is designated Cwp84 |
| <400> | 1 |

```
Met Arg Lys Tyr Lys Ser Lys Lys Leu Ser Lys Leu Leu Ala Leu
1 5 10 15
Leu Thr Val Cys Phe Leu Ile Val Ser Thr Ile Pro Val Ser Ala
                                20 25 30
Glu Asn His Lys Thr Leu Asp Gly Val Glu Thr Ala Glu Tyr Ser
                                35 40 45
Glu Ser Tyr Leu Gln Tyr Leu Glu Asp Val Lys Asn Gly Asp Thr
                                50 55 60
Ala Lys Tyr Asn Gly Val Ile Pro Phe Pro His Glu Met Glu Gly
                                65 70 75
Thr Thr Leu Arg Asn Lys Gly Arg Ser Ser Leu Pro Ser Ala Tyr
                                80 85 90
Lys Ser Ser Val Ala Tyr Asn Pro Met Asp Leu Gly Leu Thr Thr
                                95 100 105
Pro Ala Lys Asn Gln Gly Ser Leu Asn Thr Cys Trp Ser Phe Ser
                                110 115 120
Gly Met Ser Thr Leu Glu Ala Tyr Leu Lys Leu Lys Gly Tyr Gly
                                125 130 135
Thr Tyr Asp Leu Ser Glu Glu His Leu Arg Trp Trp Ala Thr Gly
                                140 145 150
Gly Lys Tyr Gly Trp Asn Leu Asp Asp Met Ser Gly Ser Ser Asn
                                155 160 165
Val Thr Ala Ile Gly Tyr Leu Thr Ala Trp Ala Gly Pro Lys Leu
                                170 175 180
Glu Lys Asp Ile Pro Tyr Asn Leu Lys Ser Glu Ala Gln Gly Ala
                                185 190 195
Thr Lys Pro Ser Asn Met Asp Thr Ala Pro Thr Gln Phe Asn Val
                                200 205 210
Thr Asp Val Val Arg Leu Asn Lys Asp Lys Glu Thr Val Lys Asn
                                215 220 225
Ala Ile Met Gln Tyr Gly Ser Val Thr Ser Gly Tyr Ala His Tyr
                                230 235 240
Ser Thr Tyr Phe Asn Lys Asp Glu Thr Ala Tyr Asn Cys Thr Asn
                                245 250 255
Lys Arg Ala Pro Leu Asn His Ala Val Ala Ile Val Gly Trp Asp
                                260 265 270
Asp Asn Tyr Ser Lys Asp Asn Phe Ala Ser Asp Val Lys Pro Glu
                                275 280 285
Ser Asn Gly Ala Trp Leu Val Lys Ser Ser Trp Gly Glu Phe Asn
                                290 295 300
Ser Met Lys Gly Phe Phe Trp Ile Ser Tyr Glu Asp Lys Thr Leu
                                305 310 315
Leu Thr Asp Thr Asp Asn Tyr Ala Met Lys Ser Val Ser Lys Pro
                                320 325 330
Asp Ser Asp Lys Lys Met Tyr Gln Leu Glu Tyr Ala Gly Leu Ser
                                335 340 345
Lys Ile Met Ser Asn Lys Val Thr Ala Ala Asn Val Phe Asp Phe
                                350 355 360
Ser Arg Asp Ser Glu Lys Leu Asp Ser Val Met Phe Glu Thr Asp
                                365 370 375
Ser Val Gly Ala Lys Tyr Glu Val Tyr Tyr Ala Pro Val Val Asn
                                380 385 390
Gly Val Pro Gln Asn Asn Ser Met Thr Lys Leu Ala Ser Gly Thr
                                395 400 405
Val Ser Tyr Ser Gly Tyr Ile Asn Val Pro Thr Asn Ser Tyr Ser
                                410 415 420
Leu Pro Lys Gly Lys Gly Ala Ile Val Val Val Ile Asp Asn Thr
                                425 430 435
Ala Asn Pro Asn Arg Glu Lys Ser Thr Leu Ala Tyr Glu Thr Asn
                                440 445 450
Ile Asp Ala Tyr Tyr Leu Tyr Glu Ala Lys Ala Asn Leu Gly Glu
                                455 460 465
Ser Tyr Ile Leu Gln Asn Asn Lys Phe Glu Asp Ile Asn Thr Tyr
                                470 475 480
Ser Glu Phe Ser Pro Cys Asn Phe Val Ile Lys Ala Ile Thr Lys
                                485 490 495
Thr Ser Ser Gly Gln Ala Thr Ser Gly Glu Ser Leu Thr Gly Ala
                                500 505 510
Asp Arg Tyr Glu Thr Ala Val Lys Val Ser Gln Lys Gly Trp Thr
                                515 520 525
Ser Ser Gln Asn Ala Val Leu Val Asn Gly Asp Ala Ile Val Asp
                                530 535 540
Ala Leu Thr Ala Thr Pro Phe Thr Ala Ala Ile Asp Ser Pro Ile
                                545 550 555
Leu Leu Thr Gly Lys Asp Asn Leu Asp Ser Lys Thr Lys Ala Glu
                                560 565 570
Leu Gln Arg Leu Gly Thr Lys Lys Val Tyr Leu Ile Gly Gly Glu
                                575 580 585
Asn Ser Leu Ser Lys Asn Val Gln Thr Gln Leu Ser Asn Met Gly
                                590 595 600
Ile Ser Val Glu Arg Ile Ser Gly Ser Asp Arg Tyr Lys Thr Ser
                                605 610 615
Ile Ser Leu Ala Gln Lys Leu Asn Ser Ile Lys Ser Val Ser Gln
                                620 625 630
Val Ala Val Ala Asn Gly Val Asn Gly Leu Ala Asp Ala Ile Ser
                                635 640 645
Val Gly Ala Ala Ala Ala Asp Asn Asn Met Pro Ile Ile Leu Thr
                                650 655 660
Asn Glu Lys Ser Glu Leu Gin Gly Ala Asp Glu Phe Leu Asn Ser
                                665 670 675
Ser Lys Ile Thr Lys Ser Tyr Ile Ile Gly Gly Thr Ala Thr Leu
                                680 685 690
Ser Ser Asn Leu Glu Ser Lys Leu Ser Asn Pro Thr Arg Leu Ala
                                695 700 705
Gly Ser Asn Arg Asn Glu Thr Asn Ala Lys Ile Ile Asp Lys Phe
                                710 715 720
Tyr Pro Ser Ser Asp Leu Lys Tyr Ala Phe Val Val Lys Asp Gly
                                725 730 735
Ser Lys Ser Gln Gly Asp Leu Ile Asp Gly Leu Ala Val Gly Ala
                                740 745 750
Leu Gly Ala Lys Thr Asp Ser Pro Val Val Leu Val Gly Asn Lys
                                755 760 765
Leu Asp Glu Ser Gln Lys Asn Val Leu Lys Ser Lys Lys Ile Glu
                                770 775 780
Thr Pro Ile Arg Val Gly Gly Asn Gly Asn Glu Ser Ala Phe Asn
                                785 790 805
Glu Leu Asn Thr Leu Leu Gly Lys
                                810
```

| | |
|---|---|
| <210> | 2 |
| <211> | 290 |
| <212> | Clostridium difficile |
| <213> | PRT |
| <220> | |
| <223> | Protein is designated FiiC |
| <400> | 2 |

```
Met Arg Val Asn Thr Asn Val Ser Ala Leu Val Ala Asn Asn Gln
1 5 10 15
Met Gly Arg Asn Val Asn Ala Gln Ser Lys Ser Met Glu Lys Leu
                                20 25 30
Ser Ser Gly Val Arg Ile Lys Arg Ala Ala Asp Asp Ala Ala Gly
                                35 40 45
Leu Ala Ile Ser Glu Lys Met Arg Ala Gln Ile Lys Gly Leu Asp
                                50 55 60
Gln Ala Gly Arg Asn Val Gln Asp Gly Ile Ser Val Val Gln Thr
                                65 70 75
Ala Glu Gly Ala Leu Glu Glu Thr Gly Asn Ile Leu Gln Arg Met
                                80 85 90
Arg Thr Leu Ser Leu Gln Ser Ser Asn Glu Thr Asn Thr Ala Glu
                                95 100 105
Glu Arg Gln Lys Val Ala Asp Glu Leu Leu Gln Leu Lys Asp Glu
                                110 115 120
Val Glu Arg Ile Ser Ser Ser Ile Glu Phe Asn Gly Lys Lys Leu
                                125 130 135
Leu Asp Gly Ser Ser Thr Glu Ile Arg Leu Gln Val Gly Ala Asn
                                140 145 150
Phe Gly Thr Asn Val Ala Gly Thr Thr Asn Asn Asn Asn Glu Ile
                                155 160 165
Lys Val Ala Leu Val Asn Thr Ser Ser Ile Met Ser Lys Ala Gly
                                170 175 180
Ile Thr Ser Ser Thr Ile Ala Ser Leu Asn Val Asp Gly Ala Ser
                                185 190 195
Gly Thr Asp Ala Ala Lys Gln Met Val Ser Ser Leu Asp Met Ala
                                200 205 210
Leu Lys Glu Leu Asn Thr Ser Arg Ala Lys Leu Gly Ala Gln Gln
                                215 220 225
Asn Arg Leu Glu Ser Thr Gln Asn Asn Leu Asn Asn Thr Ile Glu
                                230 235 240
Asn Val Thr Ala Ala Glu Ser Arg Ile Arg Asp Thr Asp Val Ala
                                245 250 255
Ser Glu Met Val Asn Leu Ser Lys Met Asn Ile Leu Val Gln Ala
                                260 265 270
Ser Gln Ser Met Leu Ala Gln Ala Asn Gln Gln Pro Gln Gly Val
                                275 280 285
Leu Gln Leu Leu Gly
                                290
```

| | |
|---|---|
| <210> | 3 |
| <211> | 506 |
| <212> | Clostridium difficile |
| <213> | PRT |
| <220> | |
| <223> | Protein is designated FliD |
| <400> | 3 |

```
Met Ser Ser Ile Ser Pro Val Arg Val Thr Gly Leu Ser Gly Asn
1 5 10 15
Phe Asp Met Glu Gly Ile Ile Glu Ala Ser Met Ile Arg Asp Lys
                                20 25 30
Glu Lys Val Asn Lys Ala Lys Gln Asp Gln Gln Ile Val Lys Trp
                                35 40 45
Lys Gln Glu Ile Tyr Arg Asp Ile Ile Lys Glu Ser Lys Asn Leu
                                50 55 60
Tyr Asp Lys Tyr Leu Asn Gly Asp Ser Pro Asn Ser Ile Thr Asn
                                65 70 75
Lys Lys Ala Tyr Ser Ala Thr Arg Ile Thr Ser Ser Asp Glu Ser
                                80 85 90
Ile Ile Val Ala Lys Gly Ser Ala Gly Ala Glu Lys Ile Asn Tyr
                                95 100 105
Gln Phe Ala Val Ser Gln Met Ala Glu Pro Ala Lys Val Thr Ile
                                110 115 120
Lys Leu Asn Ser Ser Asp Pro Ile Val Gln Gln Phe Pro Pro Asn
                                125 130 135
Ala Ser Gly Ala Ser Ser Leu Asn Ile Gly Gly Val Asn Ile Pro
                                140 145 150
Ile Ser Glu Gln Asp Thr Thr Ser Thr Ile Val Ser Lys Ile Asn
                                155 160 165
Ser Leu Cys Ala Asp Asn Asp Ile Arg Ala Ser Tyr Ser Glu Met
                                170 175 180
Thr Gly Glu Leu Ile Ile Ser Arg Lys Gln Thr Gly Ser Ser Ser
                                185 190 195
Asp Ile Asp Leu Lys Val Ile Gly Asn Asp Ser Leu Ala Gly Gln
                                200 205 210
Ile Ala Ser Asp Asn Gly Ile Thr Phe Thr Thr Asp Ala Ser Gly
                                215 220 225
Thr Lys Ser Ala Val Val Tyr Gly Lys Asn Leu Glu Ala Asp Val
                                230 235 240
Thr Asp Asp Gin Gly Arg Val Thr His Ile Ser Lys Glu Gln Asn
                                245 250 255
Ser Phe Lys Ile Asp Asn Ile Asp Tyr Asn Val Asn Ser Lys Gly
                                260 265 270
Ser Ala Lys Leu Val Ser Val Thr Asp Thr Glu Glu Ala Thr Lys
                                275 280 285
Asn Met Lys Ala Phe Val Asp Asp Tyr Asn Ala Leu Met Asp Lys
                                290 295 300
Val Tyr Gly Leu Val Thr Thr Lys Lys Ser Lys Asp Tyr Pro Pro
                                305 310 315
Leu Thr Asp Glu Gln Lys Asp Asp Met Thr Thr Glu Glu Ile Glu
                                320 325 330
Lys Trp Glu Lys Lys Ala Lys Glu Gly Ile Leu Arg Asn Asp Asp
                                335 340 345
Glu Leu Arg Ala Phe Val Glu Asp Ile Gln Ser Met Phe Phe Gly
                                350 355 360
Asp Ala Asp Thr Ile Ile Ala Leu Arg Lys Leu Gly Ile Ser Glu
                                365 370 375
His Glu Asn Tyr Asn Lys Lys Gly Gln Ile Ser Phe Asn Ala Asp
                                380 385 390
Thr Phe Ser Lys Ala Leu Ile Asp Asp Ser Asp Lys Val Tyr Lys
                                395 400 405
Ala Leu Ala Gly Tyr Ser Ser Asn Tyr Asp Asp Lys Gly Met Phe
                                410 415 420
Glu Lys Leu Lys Lys Ile Val Phe Glu Tyr Ser Gly Ser Ser Ala
                                425 430 435
Ser Lys Leu Thr Lys Lys Ala Gly Met Glu Asn Ser Ser Ser Ala
                                440 445 450
Ser Gln Asn Val Tyr Ser Lys Gln Ile Ala Glu Gln Glu Arg Asn
                                455 460 465
Ile Ser Arg Leu Val Glu Lys Met Asn Asp Lys Glu Lys Arg Leu
                                470 475 480
Tyr Ala Lys Tyr Ser Ala Leu Glu Ser Leu Leu Asn Lys Tyr Ser
                                485 490 495
Ser Gln Met Asn Tyr Phe Ser Gln Ala Gln Gly
                                500 505
```

1. An antibody composition comprising at least one IgY egg yolk component, selected from group consisting of anti-Cwp84 IgY, anti-FliC IgY, and anti-FliD IgY, each said IgY egg yolk component being produced by injecting chickens separately with a different antigen, said antigens being the proteins, Cwp84, FliC, and FliD of C. difficile.

2. An antibody composition of claim 1, wherein Cwp84 has the aminoacid sequence of Sequence No. 1, and
FliC has the aminoacid sequence of Sequence No. 2, and
FliD has the aminoacid sequence of Sequence No. 3.

3. An antibody composition of claim 2, wherein said IgY component comprises anti-FliD IgY

4. An antibody composition of claim 3, wherein said IgY component is purified anti-FliD IgY.

5. An antibody composition of claim 3, wherein said IgY component is comprising anti-FliD IgY in natural egg yolk.

6. An antibody composition of claim 2, wherein said IgY component comprises anti-Cwp84 IgY.

7. An antibody composition of claim 2, wherein said IgY compomnent is a mixture of anti-Cwp84 IgY, anti-FliC IgY, and anti-FliD IgY.

8. An antibody composition of claim 7, wherein said anti-Cwp84 IgY, anti-FliC IgY, and anti-FliD IgY have approximately equal antibody titers.

9. A method of treatment of lower mammals having a C. difficile infection, comprising administering an antibody composition comprising at least one IgY egg yolk component, selected from group consisting of anti-Cwp84 IgY, anti-FliC IgY, anti-FliD IgY, each said IgY egg yolk component being produced by injecting chickens separately with a different antigen, said antigens being the proteins, Cwp84, FliC, and FliD of C. difficile.

10. A method of claim 9, wherein Cwp84 has the aminoacid sequence of Sequence No. 1, and
FliC has the aminoacid sequence of Sequence No. 2, and
FliD has the aminoacid sequence of Sequence No. 3.

11. A method of claim 10, wherein said IgY component comprises anti-FliD IgY.

12. A method of claim 11, wherein said IgY component is purified anti-FliD IgY.

13. A method of claim 11, wherein said IgY component is purified anti-FliD IgY in natural egg yolk.

14. A method of claim 10, wherein said IgY component comprises anti-Cwp84 IgY.

15. A method of claim 10, wherein said IgY compomnent is a mixture of anti-Cwp84 IgY, anti-FliC IgY, and anti-FliD IgY.

16. A method of claim 15, wherein said anti-Cwp84 IgY, anti-FliC IgY, and anti-FliD IgY have approximately equal antibody titers.
